(19) United States
(12) Patent Application Publication
(10) Pub. No.: US 2006/0188550 A1
(43) Pub. Date: Aug. 24, 2006

(54) Method of production of animal nutritional product that increases weight gain and reduces diarrhea morbidity, mortality and severity by stimulation of natural immune response

(76) Inventor: Robert Terrell Winn, Big Sandy, TX (US)

Correspondence Address: Robert Terrell Winn DVM, 140 PR 3180, Big Sandy, TX, 75755, US

(21) Appl. No.: 11/405,844

(22) Filed: Apr. 17, 2006

Related U.S. Application Data
(63) Continuation of application No. 10/923,313, filed on Aug. 23, 2004.
(60) Provisional application No. 60/672,426, filed on Apr. 19, 2005.

(51) Int. Cl. A61K 31/716; A61K 31/715; A61K 31/714; A61K 31/198; A61K 31/4415; A61K 31/455; A61K 31/525; A61K 31/51; A61K 31/355

(52) U.S. Cl. 424/442; 424/638; 424/641; 424/702; 514/52; 514/54; 514/168; 514/251; 514/276; 514/350; 514/355; 514/458; 514/561

(57) ABSTRACT

A process of producing, collecting, increasing the volume, processing, standardizing, stabilizing, sterilizing and drying bacterial polysaccharides while retaining the activity of the contained bacterial polysaccharides is described. Nutritional compositions including the resulting product, probiotics, nutricines, vitamins, minerals, an amino acid, and a monosaccharide are depicted. The use of the product containing bacterial polysaccharides or the resulting nutritional composition the first few days of young animals' lives result in decreased diarrhea morbidity, severity and mortality. The nutritional composition also helps supply nutrients for the support of natural immune response and function.

### CROSS-REFERENCES

This application is a continuation of application Ser. No. 10/923,313 filed Aug. 23, 2004 and of the provisional Patent Application 60/672,426 filed Apr. 19, 2005, the entire contents of which are herein incorporated by reference.

### FEDERALLY SPONSORED RESEARCH OR DEVELOPMENT

The development and research for this invention involved no federal or state funding. It was supported in full by private funding.

### COMPACT DISCS AND ELECTRONIC DATA

There are no electronic data or compact discs included with this submission.

### DETAILED DESCRIPTION AND SPECIFICATION

1. Field of the Invention

The present invention relates to the collection of rumen ingesta and propagation of the bacterial flora in that ingesta to increase the amount of bacterial polysaccharides contained therein. The intent of this improvement is to maintain the diversification of the bacterial flora and the physical characteristics of the bacteria and their polysaccharide coating while allowing increased production per donor animal unit. This in turn will make the product more cost effective to the producer purchasing the product.

2. Background of the Invention

Animals are raised in concentrated rearing units. These units are used on a constant basis resulting in a build up of contamination and disease organisms. The young newborn animals are frequently affected with diarrhea. Although management practices to maximize the neonate's passive immunity are used and sanitation measures followed to minimize the exposure of newborns to virulent organisms, the diarrheal disease process is the most costly disease process affecting the rearing of young animals.

There is both a political move and a public health concern with the use of antibiotics as feed additives. To maintain health and increase productivity without the use of antibiotics is the goal of many endeavors at this time (Donovan, D. C., et al, Growth and Health of Holstein Calves Fed Milk Replacers Supplemented with Antibiotics or Enteroguard, 2002, J. Dairy Sci. 85:947-950: Webb, P. R., et al, Addition of fructooligosaccharide (FOS) and sodium diacetate (SD) plus decoquinate (D) to milk replacer and starter grain fed to Holstein calves, 1992, J. Dairy Sci. Vol 75 Suppl. 1:300). As such, there are many studies and products, which attempt to increase the immuno-competence of the neonate.

Feeding of ingesta from the rumen of adults to calves has been reported by Pounden, W. D. and Hibbs, J. W., Rumen Inoculations in Young Calves, 1949, J. American Vet. Med. Assoc., 114:33-35; Pounden, W. D. and Hibbs, J. W., The Influence of the Ration and Rumen Inoculation on the Establishment of Certain Microorganisms in the Rumens of Young Calves, 1948, J. Dairy Science, 31:1041-1050; Hibbs, J. W. and Pounden, W. D., The Influence of Pasture and Early Rumen Development on the Changes in the Plasma Carotenoids, Vitamin A and Ascorbic Acid and the Liver Storage of Carotenoids and Vitamin A of Young Dairy Calves, 1949, J. Dairy Science, 32, 1016-1024; Pounden, W. D. and Hibbs, J. W., The Influence of Pasture and Rumen Inoculation on the Establishment of Certain Microorganisms in the Rumens of Young Calves, 1949, J. Dairy Science, 32:1025-1031; Muscato, T. V., L. O. Tedeschi, and J. B. Russell, The Effect of Ruminal Fluid Preparations on the Growth and Health of Newborn, Milk-Fed Dairy Calves, 2002, J. Dairy Sci., 85:648-656 and Muscato, et al. U.S. Pat. No. 6,296,879. Feeding of dried paunch material mixed with dissolved air floatation product is described in U.S. Pat. No. 6,805,897 as a method of utilizing a waste product material for an animal feed source. Rumen contents (Paunch Product, Dehydrated) are obtained at slaughter plants and have been listed in the AAFCO manual, 1996, page 234 as an approved animal feed ingredient since 1970. The first time I was aware of it being used for feeding other cattle was in 1976.

Vaccines, serum immunoglobulins, colostrum replacers and colostrum antibody preparations have all been used to improve the neonate's immune status. Other nutritional supplements have been described. U.S. Pat. No. 6,667,063 B2 describes a composition containing as the essential ingredients colostrum, a selected whey product and defined amounts of selenium or an organic or inorganic, water soluble selenium precursor. The goal, ingredients and method of action are different from the present composition.

U.S. Pat. No. 5,374,425 describes the manufacture of a killed lactobacillus probiotic. The stabilization process is somewhat similar to the process used in the current invention. Both products are autoclaved to kill the bacterial cells. In the current invention, autoclaving takes place at 116° C. for 45-60 minutes at a pressure of 10 p.s.i. The referenced patent uses a variable temperature (100° to 121° C.) and a shorter duration (15-30 minutes). Also there is also a difference in drying. To separate the bacteria cells in U.S. Pat. No. 5,374,425 a flocculating agent is added to the culture and the cells are allowed to settle out. The liquid is decanted off. Heat, spray or freeze-drying is promoted as acceptable drying methods and the use of a drying agent is proposed. These methods except for freeze-drying are not acceptable in the current invention. Another difference is that the current patent uses rumen ingesta microorganisms, including multiple bacterial species, fungi and protozoa; while this patent uses a specific bacterial culture or mixes of dried specific bacterial cultures. U.S. Pat. No. 4,021,303 also produces killed organisms. This process includes chemically treating the microorganisms with alkali at a pH of 10.5-12.9 and a temperature of 0°-30° C., washing with water and mechanically rupturing the bacteria at a pH of 7-10.2.

There are two U.S. Pat. Nos. 5,451,400 and 5,252,329, which discuss obtaining bacteria from the ceacum of chickens to produce a probiotic product for use in chicks for the prevention of Salmonella and Campylobacter. Although this is completely different in most aspects, it is the same idea of collecting bacteria from the digestive tract to use to prevention of Gram-negative infections in young animals.

Another aim of this invention is to increase the natural local immune response by the exposure of the gut to bacterial polysaccharides in a measured, safe and controlled manner. Cell free rumen fluid has been shown to increase growth rate in calves, decrease morbidity, mortality and use of treatments for diarrheal disease (Muscato, T. V., L. O. Tedeschi, and J. B. Russell, The Effect of Ruminal Fluid Preparations on the Growth and Health of Newborn, Milk-Fed Dairy Calves, 2002, J. Dairy Sci., 85:648-656). This indicates that neither the bacterial cells nor the bacterial protein are important in and of themselves in protecting the calves. Rumen fluid has been shown to contain bacterial polysaccharides. I consider these bacterial polysaccharides are considered the “active ingredient” in rumen fluid. Bacterial polysaccharides have been shown to elicit localized immunity. The morphologic structure of the lipopolysaccaride that makes up the Gram (-) bacterial cell wall is affected by its chemical makeup and the bacteria's environment. Endotoxin morphologic structure is similar to that of the bacterial outer membrane (Brogden, K. A. and M. Phillips, The Ultrastructural Morphology of Endotoxins and LPSs., 1988, Electron Microsc. Rev., 1: 261-278). Rumen bacteria have been reported to have extracellular polysaccharide “coats” that are similar to those found on many Gram (−) organisms (Costerton, J. W., H. N. Damgaard and J. K. Cheng, Cell envelope morphology of rumen bacteria, 1974, J. of Bacteriology, 118:1132-1143). It is my belief that this similarity is the reason rumen bacteria and the environment they grow in are the best to use for this desired result.

McGruder, E. D. and G. M. Moore, in Use of lipopolysaccharide (LPS) as a positive control for the evaluation of immunopotentiating drug candidates in experimental avian colibacillosis models, 1998, Research in Veterinary Science, 66: 33-37 reported that subcutaneous administration of LPS to one-day-old broiler cockerels caused a significant reduction in all parameters of colibacillosis examined. They further state “Lipopolysaccharide (LPS) is a non-protein component of the outer membrane of Gram-negative bacteria that is devoid of direct antibacterial activity. However, it is widely known to enhance antibacterial functions of monocytic phagocytes (macrophages) via immuno-potentiation in avian species.” Clements, et al. In U.S. Pat. No. 6,436,407 describes an enterotoxin from E. coli that is described as a mutated heat-labile enterotoxin which has lost its toxicity but has retained its immunologic activity. This finding, combined with the statements in the previous paragraph, I feel further substantiates my theory.

It is theorized in U.S. Pat. No. 6,296,879 that the active ingredient in rumen fluid is bacterial protein or feed protein since this could act as an antigen to the gut. My work with chicks, showed that an overdose of the ruminal bacterial polysaccharides could have a detrimental effect. Other workers, using gram negative endotoxin have confirmed this. Jones, C. A. et al., Influence of Age on the Temperature Response of Chickens to Escherichia coli and Salmonella typhimurium Endotoxins. 1983, Poultry Science, 62:1553-1558 reported temperature decreases and following increases in young chicks following treatment with 100 μg/kg endotoxin. Webel, D. M., et al, Lipopolysaccharide-Induced Reductions in Food Intake Do Not Decrease the Efficiency of Lysine and Threonine Utilization for Protein Accretion in Chickens, 1998, J. Nutr. 128:1760-1766 reports that young chicks administered 100 or 400 μg lipopolysaccharide daily or every other day had decreased weight gain and food consumption. Most research is done with IV or Subcutaneous administration of endotoxin. Although this gives us some idea of toxicity, there is a need to convert the dosages to oral doses. Gans, H. and K. Matsumoto, Are enteric endotoxins able to escape from the intestine?, 1974, Proceedings of the Society for Experimental Biology and Medicine 147:736-739 report that a small amount of orally administered endotoxins are absorbed. This small percentage of absorption combined with the level of bacterial polysaccharides being fed in my preliminary chicken trials was above the level of LPS shown to cause negative reactions in reported studies.

Su, S. D. et al., Analysis of the immune response to lipopolysaccharide. Existence of an interspecies cross-reactive idiotype associated with anti-lipid A antibodies, 1990, Journal of immunology, 145:2994-3001 report that an antibody was produced that was protective against gram-negative bacteremia. This reveals that immunologic agents that affect gram-negative organisms may be interspecies in their action and can protect against endotoxins.

We are taught in U.S. Pat. No. 6,444,210 B1 that bacterial polysaccharides have been used as vaccines to enhance specific humoral immunity and in the particular invention named they are used to enhance general cellular immunity against a wide variety of microorganisms. The mentioned patent describes a method of isolation, purification, stabilizing and using Brucella abortus and Yersinia enterocolitica outer polysaccharide as an immunizing agent. This differs from the current invention in that the current invention makes no strides toward selecting, isolating or purifying a particular polysaccharide considered effective as an immune modulator. It further differs from the current invention in that the current invention makes no effort toward selecting, isolating or purifying the bacterial polysaccharide from the rest of the ingredients in the rumen fluid, except for excluding physically large fibers and particles. Also, the number of species of bacteria in the rumen is great and there are no steps taken to reduce this number of species. Three other similar claims have been made for specific extracts of polysaccharides to be used as vaccinal agents, see U.S. Pat. Nos. 4,210,641; 6,007,818; and 6,045,805. Another, U.S. Pat. No. 6,890,541 uses an extract of Mycobacterium phlei to elicit an immune response in young animals. The current invention differs from these four inventions for the aforementioned reasons.

We are told in U.S. Pat. No. 6,087,342 that the extraction of polysaccharides that have immune stimulating properties results in small fragments of the longer chain immune-stimulating polysaccharides. These fragments that occur have lower bioactivity than that found in the parent substance. This patent involves the use of a special substrate to bind the small fragments which potentates the activity of the fragments. This differs from the current invention in two main aspects. First an isolated product in the form of bacterial polysaccharides or bacterial nucleic acids from bacteria is used. Second this is bound to a specialized substrate. My invention uses the whole rumen fluid based bacterial culture, as it were. I also use the rumen ingesta particles and nutrient substrate particles smaller than 2 mm as the substrate that is used to carry the bacteria.

Another novel method of stimulating the immune system with bacterial produced products is described in U.S. Pat. No. 5,840,318. This method consists of growing bacteria in a stressed manner to increase the stress response factors production of the bacteria. These products are then isolated and used to activate and modulate circulating macrophages. This differs from the current invention in several methods, but primarily due to the fact that the bacteria are stressed instead of grown to peak growth rates. The stress response factors desired by the described method are not a consideration in the current invention.

Bacterial polysaccharides are produced under several patents for use as food thickeners. These patents use bacteria of the genus Xanthomonas, Leuconostoc, or Azotobacter and describe a process to grow the bacteria using specialized media or growing conditions. These descriptions are found in U.S. Pat. Nos. 3,328,262; 3,391,061; 3,433,708; 3,960,832; 3,933,788; 4,692,408 and 4,877,634. Other bacterial polysaccharides are produced for use as viscosity regulators used in various manufacturing processes as described in U.S. Pat. No. 4,567,140. A polysaccharide which forms an elastic gel and is produced by a Rhizobium sp is described in U.S. Pat. No. 6,344,346.

Rumen fluid fed fresh has resulted in increased growth rate in calves, decreased morbidity, mortality and use of treatments for diarrheal disease (Pounden, W. D. and Hibbs, J. W., The Influence of Pasture and Rumen Inoculation on the Establishment of Certain Microorganisms in the Rumens of Young Calves, 1949, J. Dairy Science, 32:1025-1031; Muscato, T. V., L. O. Tedeschi, and J. B. Russell, The Effect of Ruminal Fluid Preparations on the Growth and Health of Newborn, Milk-Fed Dairy Calves, 2002, J. Dairy Sci., 85:648-56). The obvious problems to using fresh rumen fluid include; the daily collection of the fluid, the chance of spreading disease, and the need to maintain a fistulated animal on each farm. Rumen fluid may be sterilized and bottled to increase storage time, Muscato, et al. U.S. Pat. No. 6,296,879. However, upon opening, the bottle must be refrigerated. Also, each farm would need to maintain the equipment to sterilize the rumen fluid.

Another problem is that there is no way to accurately measure the bacterial polysaccharide content of the rumen fluid daily on the farm. It has been shown that the number of rumen bacteria are affected by time of day, diet, time following feeding, location of sampling and diet physical characteristics (Bryant, M. P., and I. M. Robinson, Effects of Diet, Time After Feeding and Position Sampled on Numbers of Viable Bacteria in the Bovine Rumen, 1968, J. Dairy Sci., 51:1950-1955; Bryant, M. P., and I. M. Robinson, An Improved Nonselective Culture Media for Ruminal Bacteria and its use in Determining Diumal Variation in Numbers of Bacteria in the Rumen, 1961, J. Dairy Sci., 44:1446-1456). The result is a varying level of rumen bacterial polysaccharide content collected. This phenomenon was observed by other workers (Leedle, J. A. Z. and R. B. Hespell, Changes of Bacterial Numbers and Carbohydrate Fermenting Groups During In Vitro Rumen Incubations with Feedstuff Materials, 1984, Journal of Dairy Science, 67:808-816).

The process of the current invention allows for the collection of rumen ingesta; growth of the bacterial flora in simulated rumen conditions; sterilization of the fluid to prevent disease spread; maximization of the bacterial polysaccharide in the fluid collected by proper mixture of feed ingredients used in the fermentation growth chambers and collection following measurement of bacterial polysaccharide content to allow standardization of the amount of bacterial polysaccharide produced and used. There are patents used to measure the level of microbial activity and the presence of live organisms in fluid, however there is not a measurement to standardize the bacterial polysaccharides in a product for dosing. For examples see U.S. Pat. Nos. 5,970,163; 6,051,394; and 6,344,332 B1.

An important goal of this invention is to make the product available in an easily storable, transportable and usable form. Dried rumen fluid was considered not to have the activity of liquid rumen fluid and therefore not to be a viable alternative (Dr. J. Russell, Personal communication, Aug. 6, 2002). Field trials by the inventor using warm forced air-dried rumen fluid on drying aids have not to given the beneficial results obtained with sterilized liquid rumen fluid. Field trials by the inventor with freeze dried sterilized rumen fluid have been shown to give results equal to those obtained with sterilized liquid rumen fluid. Trials are either planned or in progress with the fermentation product, both as a liquid and a freeze-dried product. This gives this invention the distinct advantages of being easily stored, transported, used and standardized for bacterial polysaccharides. For some applications, a liquid fermentation product will be more advantageous and will also decrease the cost of the product. Standardization of the product may be made by testing and further incubation until the standard level is reached or by adding dried fermentation product to obtain the desired level of polysaccharide. Over production of polysaccharides is easily dealt with by addition of water to the fermentation solution to dilute it to the desired strength.

This product may be administered orally to individual animals by either drenching or dosing with a solution of the product. It may be fed to individual animals by mixing it into the milk fed to that animal. As taught in U.S. Pat. No. 6,733,759 B2, a specialized method of feeding the newborn chicks should be used. The ingredients used in this product may be administered to poultry by using the unique delivery system described in the aforementioned patent or a similarly devised delivery system. It may be fed as a water additive for poultry or other animals. Or, it may be top-dressed on dry feed for swine and poultry. The feeding period will range from 3-7 days and should start on day 1 or 2 of life.

### SUMMARY OF THE INVENTION

A process of producing, collecting, increasing the volume, processing, stabilizing, sterilizing and drying bacterial polysaccharides. These dried or liquid fermentation bacterial polysaccharides may be used exclusively or included into a novel composition. Such composition may contain any combination of vitamins, probiotics, minerals, an amino acid and a monosaccharide along with the bacterial polysaccharides. The invention by itself or as a composition is used to reduce the effect of diarrhea and improve performance in young animals. The present invention when used for the first few days of life is capable of reducing the severity, incidence and mortality from diarrhea. It also results in increased body weight gain.

### DRAWINGS

There are no drawings.

### SPECIFICATION

The invention Improved method of Production of Animal Nutritional Product that Increases Weight Gain and Reduces Diarrhea Morbidity, Mortality and Severity by Stimulation of Natural Immune Response is actually a method, process and composition of matter. The first section will be the process by which rumen fluid is harvested to protect and preserve the microbial population within it. It will also contain the steps necessary for production of the polysaccharide and finally sterilizing the product and rendering it suitable for storage and distribution.

Process

The animal used for collection of rumen microflora should be placed on a special diet to increase the stability of the growing conditions within the rumen and thereby increase the level of rumen bacteria. This should be done a minimum of two (2) weeks prior to the actual time of onset of rumen ingesta collection. This portion of the process will allow the rumen bacteria to adjust to the new feed and increase in numbers and growth rate. This will also allow the rumen contents to gain the actual texture necessary to allow fluid collection without having to either remove dry ingesta, add water to the rumen ingesta or remove the ingesta and rinse it with water to “wash out” the bacteria. The diet formulation, physical size and feeding regularity are all important in regulation of the rumen mat, which in turn affects the ease of collection of rumen ingesta and the growth of bacteria.

Feeding should be done a minimum of two times a day. The timing of the feedings should be such that the major feeding period is done 12 hours prior to collection and a secondary feeding with grain done 4-8 hours prior to collection. The feedings should be coincided such that the collections may be made between 1100 and 1500 hours. This timing results in a near peak production of rumen bacteria (Bryant, M. P., and I. M. Robinson, Effects of Diet, Time After Feeding and Position Sampled on Numbers of Viable Bacteria in the Bovine Rumen, 1968, J. Dairy Sci., 51:1950-1955; Bryant, M. P., and 1. M. Robinson, An Improved Nonselective Culture Media for Ruminal Bacteria and its use in Determining Diumal Variation in Numbers of Bacteria in the Rumen, 1961, J. Dairy Sci., 44:1446-1456). Multiple feedings keep the bacterial mass growing and near the peak concentration, thereby allowing for larger collections of bacterial populations. Multiple feedings also prevent large swings in bacterial growth that would influence the types and numbers of bacterial available. It is important that multiple species are available for collection to give the widest range of possible bacterial polysaccharide attachments for the neonate's gut to recognize and to help stimulate immunity.

The total amount collected is approximately one to three (1-3) gallons of rumen ingesta per collection period. A collection period is defined as the actual collection of fluid ingesta by withdrawal of fluid through a collection tube. The collection is made by first removing the cap on the rumen cannula. The hose is introduced into the rumen by first “clearing” a path with a sleeved arm. This accomplishes two goals. One is to allow the passage of the collecting hose through the rumen mat so that it may reach the fluid level. The second is to free up liquid in the mat that contains a higher level of bacteria. The hose end is covered with your hand when introducing into the rumen down to the level of the rumen fluid. The hose used may be solid or have small holes in the wall in the last six-inches (6″) of the inserted end. Vacuum is used to withdraw the fluid. In addition to the collection of the rumen fluid, some of the rumen mat may be collected to be mixed with the fluid. Generally this should be approximately ⅓ or less of the total collection. This approach allows for the greatest diversity in number of organisms collected.

A maximum of one collection period per day is performed. This allows the donor animal time to readjust rumen fluid level, consume more feed to replenish what has been removed, and to readjust electrolytes that may be affected by removing rumen fluid without the lower gut having the chance to regain nutrients that are contained within.

As the rumen fluid is removed, it should be collected into a stainless steel, glass or specially designed hard plastic receptacle to prevent any reaction between the fluid and the receptacle. The receptacle should be clean, disinfected or sterilized and rinsed with de-ionized or distilled water. This physical composition requirement and cleaning methodology will be the same for all of the numerous processing receptacles and utensils.

The receptacle should be pre-warmed to 39° C. and should be insulated. The best method of insulation would be a water bath to allow both dissipation and absorption of heat by the rumen ingesta. This will prevent any thermal damage to the bacterial flora, and should be done as to retain the diversity of the flora as much as possible. Collection of the rumen fluid should be followed immediately by inoculation of the fluid into the growth media. The growth media should be warmed to 39-40° C. prior to inoculation. The growth media is comprised of normal animal feedstuffs. The growth media should contain alfalfa as the highest percentage ingredient. The other energy products should be high fiber by-products such as soybean hulls, citrus pulp, beet pulp or corn gluten feed. The same types of feed should be fed to the donor animal at least two weeks prior to collection. Yeast culture should be added into the growth medium and into the donor's ration. Trace minerals, macro-minerals and vitamins A, D & E are also added to the growth medium.

The growth media, inoculum, artificial saliva and water should be mixed in a ratio approximating 40-80 grams, 100 ml, 100 ml and 450 ml. The growth solution should be maintained at 39-40o C during incubation. The pH should be maintained at pH 6.5±0.1 by addition of acids or buffers. The growth period should be between 4 and 24 hours. Carbon dioxide should be bubbled through the mix during the entire incubation period. Intermittent slow mixing will be required to break down bubbles and to keep the feed ingredients from stratifying. A cover capable of releasing pressure is needed to maintain an anaerobic atmosphere. This description of the technique is meant to be an illustration of the principles of the invention. The techniques used are ones that are well known to those of ordinary skill in the art.

The level of polysaccharides contained in the fermentation solution is tested hourly starting at 4 hours after initial inoculation of the solution. Once it meets the desired level of polysaccharides, the incubation period is considered finished.

Once the incubation period is finished, the fluid is then sieved through a series of sieves, starting with the largest size holes first and progressing to the smallest. The final size sieve should have holes a maximum of two (2) millimeters in diameter. The final solution will contain a slight amount of sediment. The solution should be mixed thoroughly enough to suspend this sediment and then metered into containers for autoclaving. The mixing process must be constant during filling of containers for autoclaving or the container must hold the total collection, or mixture of collections. This step is necessary to allow for testing and standardization of the bacterial polysaccharide if the final product. Following autoclaving of the standardized fermentation solution, it is ready to use as a liquid product. Should drying be desired or further processing for example by addition of other ingredients in a sterile manner or repackaging, this will be done following sterilization.

The autoclaving process should be started immediately to prevent excess gas formation within the container that will prevent the sealing of the container. Allowing the fluid to incubate for a period of time prior to autoclaving may increase the bacterial population, however, it may also change the population (Wells, J. E. and J. B. Russell, Why Do Many Ruminal Bacteria Die and Lyse so Quickly?, 1996, J. Dairy Sci. 79:1487-1495). This probable change in population has not been studied nor the results of the resulting product tested.

These containers are labeled to allow control of each collected lot. Each lot collected must be tested for bacterial polysaccharides. Therefore it is important to keep each lot identified the same. In addition, each lot autoclaved must be tested for bacterial growth, both aerobic and anaerobic. It then becomes necessary that each lot autoclaved be identified, regardless of the incubation lot from which it originated. Autoclaving should be done for a period of 45-60 minutes at of temperature of 240° F. (116° C.) and 10 pounds per square inch of pressure.

Following autoclaving, the rumen fluid fermentation product is allowed to cool. Freeze-drying may be started immediately, it may be stored for variable amounts of time prior to further processing or it may be used as a liquid. Prior to freeze-drying, samples from each collection lot is taken to be tested for bacterial polysaccharides. In addition, each lot autoclaved must be sampled and tested for bacterial growth, both aerobic and anaerobic growth. These samples must be taken prior to freeze-drying, but the testing does not have to be finished prior to freeze-drying. Each lot of freeze-dried material must be labeled with the incubation lot and the autoclaved lot. The amount of material from each incubation lot placed into each freeze-drying tray or lot must be recorded.

The trays are first frozen in a freezer that is not frost-free and then placed into the freeze-drying chamber. The freeze-drying process to be followed is in the following schedule. It is probable and expected that the schedule will change from one type of freeze-drying equipment to another. In addition the depth of the trays and the amount of liquid used will also affect the drying time and results.

| Day | Set Point | Vacuum millitors |
|---|---|---|
| 1 | (−) 20° F. | 103 |
| 2 | (−) 15° F. | 104 |
| 3 | (−) 10° F. | 106 |
| 4 | (−) 5° F. | 99 |
| 5 | 0 | 102 |
| 6 | 10° F. | 87 |
| 7 | 20° F. | 90 |
| 8 | 30° F. | 94 |
| 9 | 40° F. | 92 |
| 10 | 50° F. | 91 |
| 11 | 70° F. | 92 |

Once the product is dried, it is removed from the chamber, scraped out of the drying tray into a mixing container. At this point it is ready to be mixed into an amino acid carrier that will be used to dilute the dried rumen fluid fermentation product and allow it to be standardized. After standardization of the freeze-dried product, it is possible to use this product to fortify a batch of liquid rumen fluid fermentation product that does not have the desired level of bacterial polysaccharides. In addition it may be used as the dry product and fed separately or mixed with vitamins, probiotics, minerals, an amino acid and a monosaccharide to form a novel composition used to reduce the effect of diarrhea in young animals.

Method

The invention Improved method of Production of Animal Nutritional Product that Increases Weight Gain and Reduces Diarrhea Morbidity, Mortality and Severity by Stimulation of Natural Immune Response as stated above is actually a method, process and composition of matter. The process of producing the raw material used in the production of the final composition of matter has been described. A description of the Method of Standardization of the product will be described. Without a method of standardizing the bacterial polysaccharides, there would be no way of producing a product that works consistently and remains the same from batch to batch.

Method of Standardization

Each lot incubated must be tested for bacterial polysaccharides. This test will quantify the amount of bacterial polysaccharides in the lot. The amount of product that is placed into the freeze dryer may then be mathematically used to determine the amount of bacterial polysaccharides found in the lot. This dried material will then be added to an amino acid carrier to allow for standardization.

The level of polysaccharides contained in the fermentation solution is tested hourly starting at 4 hours after initial inoculation of the solution. Once it meets the desired level of polysaccharides, the incubation period is considered finished and the product is considered standardized if it contains within 0-10% more polysaccharide than the desired level. Should more that 10% of the level of bacterial polysaccharides desired be contained in the tested product, it will be standardized as is described below.

For example, if the incubated fluid prior to drying contained 300 μg hexose equivalent bacterial polysaccharides/ml. and there were 4000 ml. dried, we simply multiply the 4000 ml.×300 μg. This would equal 1,200,000 μg of bacterial polysaccharides in the total wafer of dried material. There must be two assumptions to continue. First, how much bacterial polysaccharide is desired per dose, and second, how large is the dose of carrier material to be used? I want to use 2,400 ug of bacterial polysaccharides per dose and I would like a 5-gram inclusion per dose. This batch of dried material contains 1,200,000 ug of bacterial polysaccharides. To determine the number of doses that may be made from this batch, divide the total μg of bacterial polysaccharides by the μg of bacterial polysaccharides desired per dose. 1,200,000 μg/2,400 μg per dose=500 doses. This is the number of doses we know we can make from this mix.

From previous experiments, 4000 ml of solution will render about 80 grams of dried product. The assumption was that there would be a 5-gram inclusion per dose. It is determined that this batch will make 500 doses. 500 doses×5 grams=2,500 grams. Now, put all of the dried material into a receptacle on a scale. The receptacle should have had the tare checked prior to the dried material being placed into it. Q. S. the total with the carrier amino acid to reach 2,500 grams net weight. This material should then be thoroughly mixed. This mixed product of the carrier amino acid and the dried rumen fermentation product will now be standardized. This standardized dried product may be used as it is, incorporated with other nutritional aids to form a novel product, or mixed back with a liquid lot to fortify the lot, simply by weight.

For example, if a liquid lot were tested prior to autoclaving and tested 250 μg hexose equivalent bacterial polysaccharides/ml. and there were 4000 ml. We know that the lot contains 1,000,000 μg hexose equivalent bacterial polysaccharides (250 μg×4000 ml=1000000). To standardize the lot (if it were to be used as a liquid) the dried fermentation product could be added prior to autoclaving. Assume that we standardized the dried product to 2400 μg per 5-gram dose. We want to raise the level of the current liquid lot to 300 μg hexose equivalent bacterial polysaccharides/ml. and there were 4000 ml. 300-250=50 μg hexose equivalent bacterial polysaccharides/ml.×4000 ml=200000 μg hexose equivalent bacterial polysaccharides. There are 2400 μg per 5-gram dose or 2400/5=480 μg per gram. 200000/480=416.7 grams. QS the total volume to 4000 ml. This will standardize the lot to 300 μg hexose equivalent bacterial polysaccharides/ml.

For example, if a liquid lot were tested prior to autoclaving and tested 1200 μg hexose equivalent bacterial polysaccharides/ml. and there were 4000 ml. We know that the lot contains 4,800,000 μg hexose equivalent bacterial polysaccharides (1200 μg×4000 ml=4800000). To standardize the lot (if it were to be used as a liquid) water could be added prior to autoclaving. Assume we want to lower the level of the current liquid lot to 1000 μg hexose equivalent bacterial polysaccharides/ml. and there were 4000 ml. The 4000 ml contains 4,800,000 hexose equivalent bacterial polysaccharides/ml./1000 μg hexose equivalent bacterial polysaccharides desired. This would give an answer of 4800 which would be the number of ml that could be produced at the level of 1000 μg hexose equivalent bacterial polysaccharides/ml. Simply QS the total volume to 4800 ml with water. This will standardize the lot to 1000 μg hexose equivalent bacterial polysaccharides/ml.

Composition of Matter

The invention Improved method of Production of Animal Nutritional Product that Increases Weight Gain and Reduces Diarrhea Morbidity, Mortality and Severity by Stimulation of Natural Immune Response is also a new composition of matter. It is not a composition in the sense of a new chemical entity, but in the sense of the same molecular structures only found in nature but now available in a produced form that is fully functional. This of course refers to the rumen fluid fermentation component of the product that includes the bacterial polysaccharides which function as well as the naturally occurring ruminal fluid bacterial polysaccharides.

In addition, the actual formulas that may include these bacterial polysaccharides along with several other specific ingredients that add to the effectiveness of the bacterial polysaccharides and the overall product result in the complete product being a new chemical composition of matter. The formulas may include any or all of the following. A specific amino acid that supports the production of mucous found in the intestinal tract. It also may contain monosaccharides, which are used to standardize the dose and supply energy without affecting the bacterial polysaccharides. Immunosupportive antioxidant products such as vitamins A and E, trace-minerals such as copper, zinc, manganese and selenium may be added to the composition. B-complex vitamins to ensure proper enzyme function may be added. Probiotics such as Lactobacilli and Bifidobacter may be added to help establish a healthy population of bacteria in the gut as well as suppress the level of pathogenic bacteria in the gut.

1. A method for the promotion of growth and weight gain, the abatement of diarrheal disease and the reduction in mortality in a farm animal comprising the administration of bacterial polysaccharides derived from anaerobic fermentation cultures, for which rumen ingesta was the inoculum, exclusively or combined with nutritional aids for the first 1-7 days of life.

2. A method according to claim 1, in which the farm animal is a calf.

3. A method according to claim 1, in which the farm animal is a pig.

4. A method according to claim 1, in which the farm animal is a chick.

5. A method according to claim 1, in which the farm animal is a turkey poult.

6. A method according to claim 1, in which the farm animal is a foal.

7. A method according to claim 1, in which the farm animal is a kid.

8. A method according to claim 1, in which the farm animal is a lamb.

9. A method according to claim 1, in which the nutritional aids contain 20-95% of the amino acid threonine.

10. A method according to claim 1, in which the nutritional aids contain 5-70% of a monosaccharide.

11. A method according to claim 1, in which the nutritional aids contain the recommended daily dose of vitamins A, D, E for the newborn of the species being fed.

12. A method according to claim 1, in which the nutritional aids contain a specially selected probiotic for the species being fed.

13. A method according to claim 1, in which the nutritional aids contain the recommended daily dose of Thiamine, Riboflavin, Pyridoxine, Pantothenic acid, Niacin, Biotin, Folic acid, B₁₂ and Choline for the newborn of the species being fed.

14. A method according to claim 1, in which the nutritional aids contain the organic trace minerals Manganese, Zinc, Copper, and Selenium.

15. A preventative feed additive composition for the promotion of growth and weight gain, the abatement of diarrheal disease and the reduction in mortality in a farm animal comprising the administration of bacterial polysaccharides derived from anaerobic fermentation cultures, for which rumen ingesta was the inoculum, exclusively or combined with nutritional aids for the first 1-7 days of life.

16. The composition in claim 15, wherein the nutritional aids contains 20-95% of the amino acid threonine.

17. The composition in claim 15, wherein the nutritional aids contains 5-70% of a monosaccharide.

18. The composition in claim 15, wherein the nutritional aids contain the recommended daily dose of vitamins A, D, E for the newborn of the species being fed.

19. The composition in claim 15, wherein the nutritional aids contains a specially selected probiotic for the species being fed.

20. The composition in claim 15, wherein the nutritional aids contain the recommended daily dose of Thiamine, Riboflavin, Pyridoxine, Pantothenic acid, Niacin, Biotin, Folic acid, B₁₂ and Choline for the newborn of the species being fed.

21. The composition in claim 15, wherein the nutritional aids contains the organic trace minerals Manganese, Zinc, Copper, and Selenium.

22. A preventative feed additive composition in claim 15, in which the intended farm animal is a calf.

23. A preventative feed additive composition in claim 16, in which the intended farm animal is a calf.

24. A preventative feed additive composition in claim 17, in which the intended farm animal is a calf.

25. A preventative feed additive composition in claim 18, in which the intended farm animal is a calf.

26. A preventative feed additive composition in claim 19, in which the intended farm animal is a calf.

27. A preventative feed additive composition in claim 20, in which the intended farm animal is a calf.

28. A preventative feed additive composition in claim 21 in which the intended farm animal is a calf.

29. A preventative feed additive composition in claim 15 in which the intended farm animal is a pig.

30. A preventative feed additive composition in claim 16 in which the intended farm animal is a pig.

31. A preventative feed additive composition in claim 17 in which the intended farm animal is a pig.

32. A preventative feed additive composition in claim 18 in which the intended farm animal is a pig.

33. A preventative feed additive composition in claim 19 in which the intended farm animal is a pig.

34. A preventative feed additive composition in claim 20 in which the intended farm animal is a pig.

35. A preventative feed additive composition in claim 21 in which the intended farm animal is a pig.

36. A preventative feed additive composition in claim 15 in which the intended farm animal is a chick.

37. A preventative feed additive composition in claim 16 in which the intended farm animal is a chick.

38. A preventative feed additive composition in claim 17 in which the intended farm animal is a chick.

39. A preventative feed additive composition in claim 18 in which the intended farm animal is a chick.

40. A preventative feed additive composition in claim 19 in which the intended farm animal is a chick.

41. A preventative feed additive composition in claim 20 in which the intended farm animal is a chick.

42. A preventative feed additive composition in claim 21 in which the intended farm animal is a chick.

43. A preventative feed additive composition in claim 15 in which the intended farm animal is a foal.

44. A preventative feed additive composition in claim 16 in which the intended farm animal is a foal.

45. A preventative feed additive composition in claim 17 in which the intended farm animal is a foal.

46. A preventative feed additive composition in claim 18 in which the intended farm animal is a foal.

47. A preventative feed additive composition in claim 19 in which the intended farm animal is a foal.

48. A preventative feed additive composition in claim 20 in which the intended farm animal is a foal.

49. A preventative feed additive composition in claim 21 in which the intended farm animal is a foal.

50. A preventative feed additive composition in claim 15 in which the intended farm animal is a turkey poult.

51. A preventative feed additive composition in claim 16 in which the intended farm animal is a turkey poult.

52. A preventative feed additive composition in claim 17 in which the intended farm animal is a turkey poult.

53. A preventative feed additive composition in claim 18 in which the intended farm animal is a turkey poult.

54. A preventative feed additive composition in claim 19 in which the intended farm animal is a turkey poult.

55. A preventative feed additive composition in claim 20 in which the intended farm animal is a turkey poult.

56. A preventative feed additive composition in claim 21 in which the intended farm animal is a turkey poult.

57. A preventative feed additive composition in claim 15 in which the intended farm animal is a kid.

58. A preventative feed additive composition in claim 16 in which the intended farm animal is a kid.

59. A preventative feed additive composition in claim 17 in which the intended farm animal is a kid.

60. A preventative feed additive composition in claim 18 in which the intended farm animal is a kid.

61. A preventative feed additive composition in claim 19 in which the intended farm animal is a kid.

62. A preventative feed additive composition in claim 20 in which the intended farm animal is a kid.

63. A preventative feed additive composition in claim 21 in which the intended farm animal is a kid.

64. A preventative feed additive composition in claim 15 in which the intended farm animal is a lamb.

65. A preventative feed additive composition in claim 16 in which the intended farm animal is a lamb.

66. A preventative feed additive composition in claim 17 in which the intended farm animal is a lamb.

67. A preventative feed additive composition in claim 18 in which the intended farm animal is a lamb.

68. A preventative feed additive composition in claim 19 in which the intended farm animal is a lamb.

69. A preventative feed additive composition in claim 20 in which the intended farm animal is a lamb.

70. A preventative feed additive composition in claim 21 in which the intended farm animal is a lamb.

71. A method of freeze-drying bacterial polysaccharides to maintain the bioactivity of the bacterial polysaccharides.

72. A method of growing rumen bacterial population in increased amounts using a cow as the growth apparatus, a special formulation of normal cow feed as a substrate and a combination of feeding time, time of day, feeding technique, collection area, feed physical form and number of feedings to maximize bacterial growth and diversity.

73. A method of standardizing the dose of bacterial polysaccharides by measuring the amount of bacterial polysaccharides in the fermentation solution, measuring the amount of fluid dried, then determining the total amount of bacterial polysaccharides contained in the dried fermentation solution wafer. This quantity of bacterial polysaccharides is then used to determine the total number of doses that may be produced. The freeze dried fermentation solution wafer is then weighed and an amino acid carrier (threonine) is added to Q. S. the total to the weight represented by the weight of the total number of doses that may be produced.

74. A method of standardizing the level of bacterial polysaccharides in the liquid fermentation product by testing during incubation and adding dried bacterial polysaccharides or water to obtain the concentration desired.

75. A method of producing, collecting, increasing the volume, processing, standardizing, stabilizing, sterilizing and drying bacterial polysaccharides while retaining the activity of the contained bacterial polysaccharides

76. A method to maintain the diversification of the ruminal bacterial flora collected and the physical characteristics of the bacteria and their polysaccharide coating while allowing increased production per donor animal unit.
